(19) United States
(12) Patent Application Publication
(10) Pub. No.: US 2010/0168613 A1
(43) Pub. Date: Jul. 1, 2010

(54) FECAL EXAMINATION RECEPTACLE

(76) Inventor: Robert J. Greenwald, Venice, FL (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/345,119

(22) Filed: Dec. 29, 2008

(51) Int. Cl. A61B 5/00

(52) U.S. Cl. 600/573

(57) ABSTRACT

A fecal examination preparation receptacle comprising a plastic transparent body having a cap which can be removed to permit fecal matter to be received in the receptacle to be mixed with a flotation material to aid in separating the fecal matter and ova or eggs of parasites that may be contained in the fecal matter and then dispensed through a filter mounted in the cap of the receptacle for examination of the filtered material for the presence of the ova of parasites.

### FIELD OF THE INVENTION

The present invention relates to a receptacle for receiving fecal matter and for aiding in the separation of the feces from ova or eggs that might be contained in the fecal matter.

### BACKGROUND OF THE INVENTION

The examination of fecal matter to determine whether or not the ova or eggs of parasites are present generally includes the step of depositing the fecal matter in a container of a solution with a specific gravity within the range of 1.22 to 1.40 and breaking up the fecal matter to separate the ova from the remaining fecal matter to float the ova to the top of the container. The separated ova then can be examined to determine the necessary treatment. Examination can be made by placing the separated material on a slide and examining the material under a microscope. This method is generally referred to as the flotation process.

In another known method the mixture is put in a centrifuge and the lighter ova is separated from the fecal matter by action of the centrifuge. Again the separated ova is examined by a microscope. This method of examination is generally referred to as the centrifuge process.

### SUMMARY OF THE INVENTION

The present invention provides a receptacle for the fecal matter and for aiding in the separation of ova from the fecal matter for use in either the flotation process or the centrifuge process. A solution of the proper specific gravity is added to the receptacle and the fecal matter and a cap is provided to close the receptacle. The solution is selected to have a specific gravity in the range of 1.22 to 1.40 to aid in causing the separated ova to rise in the solution when used in the floatation method or the centrifuge method of examination. The receptacle of the present invention is useful in either of these methods of examination in providing the ova in the proper form for examination by the use of either method.

The receptacle is constructed of a pliable, transparent material that permits the sides of the receptacle to be squeezed together to break up the fecal matter. The transparent sides permit monitoring the mixing. The cap is provided with a spout and in one form of the invention a breakaway opening in the spout. A filter is mounted in the cap so that pouring the contents from the receptacle will permit passage of the ova and smaller particles of fecal matter but will prevent the larger remaining fecal matter from being poured from the receptacle. The ova then can be further separated from the remaining matter and examined by using either the flotation method or the centrifuge method. This provides a fecal examination system in which direct contact with the fecal matter is not necessary prior to the actual examination thus substantially eliminating unpleasant contact by humans during the process.

### BRIEF DESCRIPTION OF THE DRAWINGS

A better understanding of the present invention will be achieved by referring to the drawings in wherein

FIG. 1 is an elevational view of the fecal receptacle of present invention;

FIG. 2 is an exploded elevational view of the receptacle shown in FIG. 1;

FIG. 3 is a partial cross sectional view taken substantially along line 3-3 of FIG. 1;

FIG. 4 is an elevational view of the present invention substantially as seen from right side of Fig.;

FIG. 5 is a view similar to FIG. 1 but illustrating another preferred construction of the fecal receptacle of the present invention; and

FIG. 6 is a view illustrating the fecal receptacle of the present invention in use practicing one step of the method of the present invention.

### DETAILED DESCRIPTION OF THE PREFERRED EMBODIMENTS

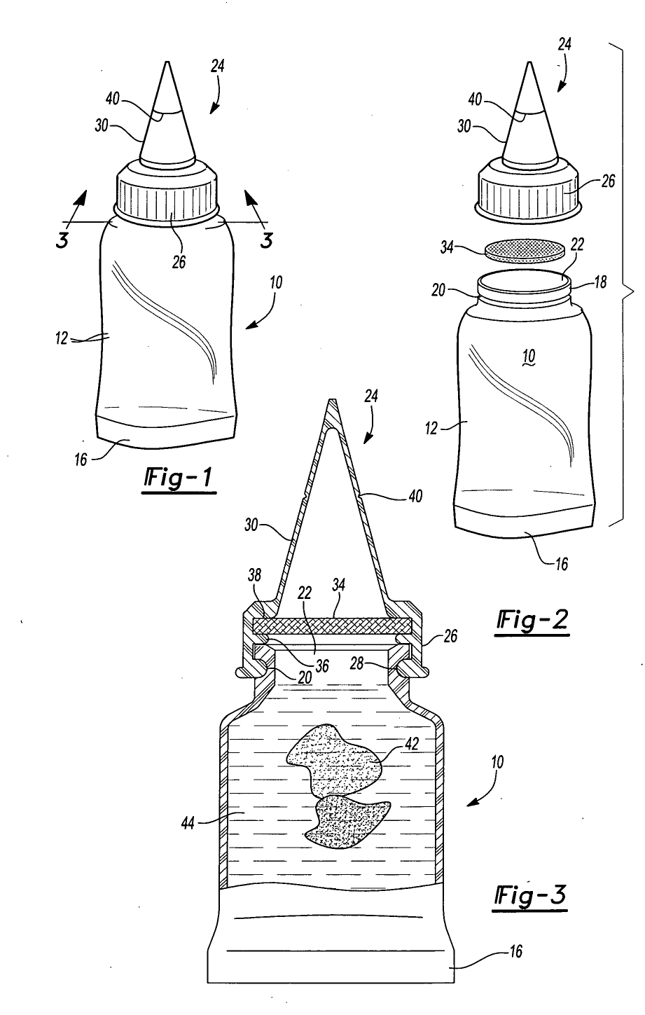

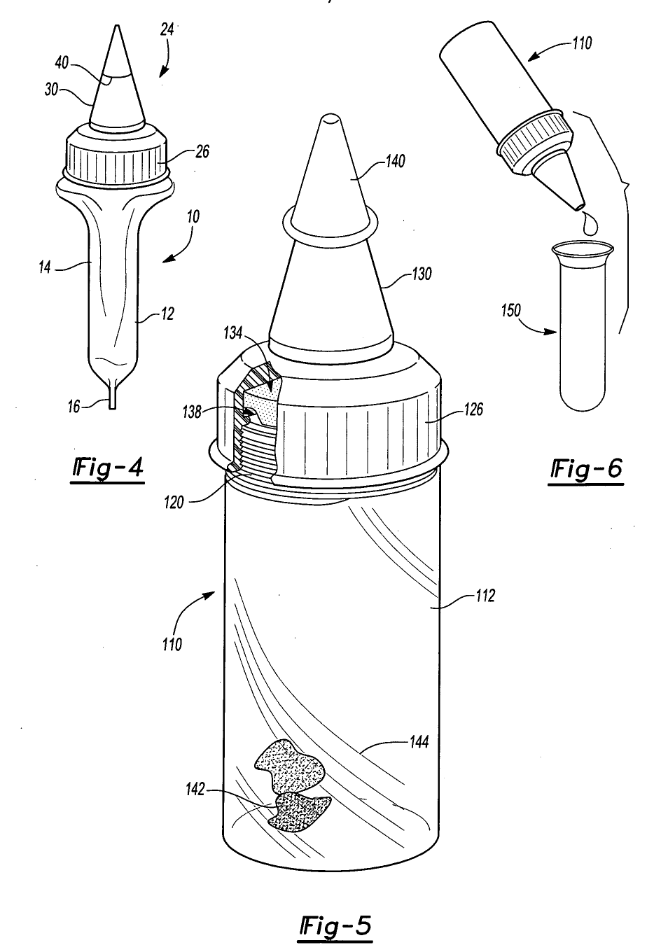

Now referring to the drawings FIG. 1 illustrates the one preferred receptacle 10 of the present invention in the form of a tube having opposing substantially flat sides 12 and 14 (FIG. 4), a sealed bottom 16 and a top 18 provided with an outer screw thread 20 (FIGS. 2 and 3) which defines an opening 22. A cap 24 is adapted to close the opening 22 by being screwed unto the thread 20.

The cap 24 includes a base 26 having internal threads 28 (FIG. 3) for receiving the threads 20 and a spout 30 that extends through an opening 32 formed in the base 26. A filter 34 is mounted in the spout 30 between flanges 36 and 38. The spout 30 is provided with a weakened section 40 to permit removal of the top portion of the spout 30 to provide for pouring the contents from the receptacle 10.

FIG. 3 illustrates the receptacle 10 broken away to more clearly illustrate the contents of fecal matter 42 and solution 44. The receptacle 10 is preferably constructed of a transparent material so that the fecal matter 42 and solution 44 will be visible through the sides 12 and 14 of the receptacle 10. To prepare for the reception of the solution 44 the base 26 is removed from the top 18 of the receptacle 10 and the solution 44 is poured into receptacle 10. The receptacle 10 is not completely filled with solution 44 but room is left for the reception of the fecal matter 42. The material used for the body of the receptacle 10 is also pliable so that the fecal matter 42 can be broken up and mixed with the solution 44 by squeezing the sides 12 and 14 of the receptacle 10 together to mash the fecal matter 42 into the solution 44.

The solution 44 provides for flotation or levitation of parasite eggs or ova and the like introduced into the solution 44 with the fecal matter 42 for a purpose that will be described later. The solution 44 can consist of sodium nitrate or sucrose, or magnesium sulfate, or zinc sulfate, sodium chloride or other solutions and the purpose of which is to raise the specific gravity to range of 1.22 to 1.40 so that the ova will rise to the top of the solution.

Once the fecal matter 42 has been broken into small pieces the weakened portion 40 of spout 30 is broken away to permit a portion of the solution 44 containing the fecal matter and ova to be poured out of the receptacle 10 to be examined. The filter 34 limits the size of the material permitted to flow from the receptacle 10 with the solution.

FIGS. 5 and 6 illustrate another preferred receptacle 110 of the present invention as being in the form of a bottle having base 112 preferably constructed of transparent pliable plastic material. The receptacle 110 is provided with an externally treaded top 120 for receiving a cap 126. The cap 126 is provided with an open top pouring spout 130 with a removable closure member 140. A filter 134 mounted with in the cap 126 filters material that will be poured from the receptacle through the spout 130. A flange 138 formed internally in the cap retains the filter in place.

Again as with the receptacle 10 the receptacle 110 is provided with a solution 144 like the solution 44 and fecal matter 142 is added to the solution 144. The base 112 is sufficiently pliable to permit squeezing of the sides of the base 112 together with the fecal matter 142 in between to break the fecal matter 142 into smaller pieces again without requiring human contact with the fecal matter 142.

FIG. 6 illustrates the solution and the filtered matter being poured into a test tube 150. Letting the solution 144 and the material within the solution 144 to sit for a period of time will cause the ova to rise in the solution where it can be examined under a microscope to determine the necessary treatment. Using this method of separating the ova from the fecal matter is using the receptacle to aid in the method commonly termed the flotation method of separation.

The test tube 150 can instead be immediately mounted to a centrifuge (not shown) to separate the ova from the fecal matter by centrifugal force. In such a method the solution, having a specific gravity greater than the ova, will facilitate the separation.

It should be apparent that although use of the receptacle of the present invention will most often be used as a part of the examination process for examining the feces of animals such as dogs and cats for the presence of worms and other parasites it will also find use in the examination of human feces.

It should also be apparent that the present invention provides a receptacle for use in the fecal matter examination process that eliminates several steps in the present process. A disposable receptacle is provided for mixing the fecal matter with the solution that does so with a minimum of exposure to the fecal matter by the examiner. An empty receptacle can be provided to the animal owner or the patient and the receptacle can be used to deliver the specimen to the examiner. The examiner can then add the solution and, after mixing, remove the top of the spout and pour out the small amount of ova and solution to permit examination. The receptacle then, with the remaining contents, can be discarded.

Although the examination process has been described as including examination of the separated matter by microscope on a slide it should be understood that the receptacle has utility in a process in which a centrifuge is used. The material can be deposited through the spout to a test tube for use in the centrifuge. With either use a receptacle has been provided for fecal examination that minimizes human contact with the material being examined.

While the receptacles 10 and 110 have been described as being constructed of a transparent material it should be apparent that a translucent material could be used to construct the sides of the receptacles as well. All that is necessary is that the fecal matter be sufficiently visible to permit locating the material to permit it to be mashed by squeezing the sides of the receptacle together.

It should also be apparent that changes could be made to the receptacle as described without departing from the spirit of the invention as set forth in the following claims.

1-8. (canceled)

9. A receptacle for receiving fecal matter to be examined comprising:
a) said receptacle having an opening for receiving fecal matter and a flotation solution;
b) a cap removably covering said opening;
c) said receptacle further having sides constructed of sufficiently pliable material to permit said sides to be squeezed together to engage and mash the fecal matter to break said fecal matter into small pieces to promote mixing said fecal matter and said flotation solution;
c) a filter disposed within said outlet to filter the mixed fecal matter and flotation solution material dispensed through said outlet.

10. The receptacle as defined in claim 9 and further comprising:
a) a cap being provided for said receptacle; said cap including a spout extending outwardly from said cap;
b) said spout having an outlet and means for opening and closing said outlet;
c) said filter being disposed within said cap between said receptacle and said outlet.

11. The receptacle as defined in claim 9 and being constructed of transparent material to permit the fecal matter within the receptacle to be viewed.

12. The receptacle as defined in claim 9 and being constructed of translucent material to permit the fecal matter within the receptacle to be viewed.

13. The receptacle as defined in claim 9 and being constructed in the form of a tube.

14. The receptacle as defined in claim 9 and being constructed in the form of a bottle.

15. A method of examining fecal matter for the presence of ova and other matter including the steps of:
a) providing a receptacle with an open top and sides; said receptacle being constructed of a sufficiently pliable material to permit the sides of the receptacle to be squeezed into contact with each other;
b) introducing fecal matter and a flotation solution into said receptacle;
c) mixing said fecal matter and said flotation solution in said receptacle by squeezing the sides of said receptacle together to mash said fecal matter therebetween;
d) dispensing the mixed fecal matter and flotation solution into a test tube to be used in a centrifugation or a flotation process.

16. The method as defined in claim 15 and including the step of filtering the mixture prior to dispensing it into a test tube.
